(19) United States
(12) Patent Application Publication
(10) Pub. No.: US 2023/0316948 A1
(43) Pub. Date: Oct. 5, 2023

(54) SYSTEM AND METHOD FOR MAINTAINING ENGAGEMENT IN A WEIGHT LOSS PROGRAM

(71) Applicant: Wendy Scinta, Cazenovia, NY (US)

(72) Inventor: Wendy Scinta, Cazenovia, NY (US)

(21) Appl. No.: 18/128,172

(22) Filed: Mar. 29, 2023

Related U.S. Application Data
(60) Provisional application No. 63/362,074, filed on Mar. 29, 2022.

(51) Int. Cl. G09B 19/00; G16H 20/60; G16H 10/60

(52) U.S. Cl. CPC G09B 19/0092; G16H 20/60; G16H 10/60

(57) ABSTRACT

A system and method for maintaining engagement in a weight loss program includes a software application stored on a patient's smart phone. The application provides for the patient to initially enter demographic, weight loss plan, and other information into a database, and then periodically update the data The stored data is processed by a processor and customized messaging is generated and transmitted to the patient's smart phone device. The data is also made available to the patient's medical provider for monitoring the patient's weight loss progress.

### CROSS-REFERENCE TO RELATED APPLICATION

This application claims priority to U.S. Provisional Patent Application Ser. No. 63/362,074, filed on Mar. 29, 2022, the entire disclosure of which is incorporated herein by reference.

### FIELD OF THE INVENTION

The present disclosure is directed generally to a system and method for maintaining engagement in a weight loss program.

### BACKGROUND

It is commonly understood, and research has shown that individuals disengage from a weight loss program because they are not seeing progress and simply cannot maintain the weight loss program. Prior to becoming disengaged, however, indicators showing the individual to be vulnerable include stress, personal or financial instability, a decrease in communication or accountability, or simple non-adherence to the plan. Prior or simultaneous with this, negative weight change occurs either through prolonged stability of weight or weight gain.

Accordingly, there is a need in the art for a system and method that will maintain engagement of an individual in a weight loss program.

### SUMMARY

The present disclosure is directed to a system and method of maintaining engagement of an individual in a weight loss program.

According to an aspect is a computer implemented method of maintaining engagement of an individual in a weight loss program, comprising the steps of inputting data into a non-transitory memory of the computer that is representative of the individual's demographics, including weight; periodically inputting data into the non-transitory memory of the computer that is representative of the individual's weight loss in units over a first predetermined time interval; periodically inputting data into the non-transitory memory of the computer that is representative of the individual's subjective motivation level on a scale graded from lowest motivation to highest motivation level over the first predetermined time interval; calculating data representative of a weight loss slope over a second predetermined interval which is greater than the first time interval; generating a score representing the individual's level of engagement in the weight loss program by summing the data representative of the individual's weight loss in units over a first predetermined time interval, the data representative of the individual's subjective motivation level on a scale graded from lowest motivation to highest motivation level over the first predetermined time interval, and the data representative of a weight loss slope over a second predetermined interval; and generating in the computer and communicating messages to the patient from the computer on a third interval, wherein the messages are customized to the patient based upon the score representing the individual's level of engagement.

According to an embodiment, the first-time interval is one week, the second time interval is four weeks, and the third interval is daily.

According to an embodiment, [the scale representative of the individual's subjective motivation level ranges from 1 to 10 wherein 1 represents the lowest possible motivation and 10 represents the highest possible motivation.

According to an embodiment, the method further comprises the step of transmitting the score representing the individual's level of engagement into a medical record of the individual.

According to an embodiment, the method further comprises the step of inputting data into a non-transitory memory of the computer that is representative of the individual's eating behaviors.

According to an embodiment, the method further comprises the step of inputting data into a non-transitory memory of the computer that is representative of the individual's nutrition.

According to an embodiment, the method further comprises the step of inputting data into a non-transitory memory of the computer that is representative of the individual's medications.

According to an embodiment, the method further comprises the step of inputting data into a non-transitory memory of the computer that is representative of the individual's sleep patterns.

According to an embodiment, the method further comprises the step of inputting data into a non-transitory memory of the computer that is representative of the individual's physical activity level.

According to an aspect is a system for maintaining engagement of an individual in a weight loss program, comprising: a non-transitory computer readable medium with instruction encoded thereon; and one or more processors programmed, structured and/or configured to, when executing the instructions, perform the operations of: receiving data into the non-transitory computer readable medium of the computer that is representative of the individual's demographics, including weight; periodically receiving data into the non-transitory computer readable medium that is representative of the individual's weight loss in units over a first predetermined time interval; periodically receiving data into the non-transitory computer readable medium that is representative of the individual's subjective motivation level on a scale graded from lowest motivation to highest motivation level over the first predetermined time interval; calculating data representative of a weight loss slope over a second predetermined interval which is greater than the first time interval; generating a score representing the individual's level of engagement in the weight loss program by summing the data representative of the individual's weight loss in units over a first predetermined time interval, the data representative of the individual's subjective motivation level on a scale graded from lowest motivation to highest motivation level over the first predetermined time interval, and the data representative of a weight loss slope over a second predetermined interval; and generating in the computer and communicating messages to the patient from the computer on a third interval, wherein the messages are customized to the patient based upon the score representing the individual's level of engagement.

According to an embodiment, the first-time interval is one week, the second time interval is four weeks, and the third time interval is daily.

According to an embodiment, the scale representative of the individual's subjective motivation level ranges from 1 to 10 wherein 1 represents the lowest possible motivation and 10 represents the highest possible motivation.

According to an embodiment, the one or more processors are programmed, structured and/or configured to, when executing the instructions, perform the further operations comprising the step of transmitting the score representing the individual's level of engagement into a medical record of the individual.

According to an embodiment, the one or more processors are programmed, structured and/or configured to, when executing the instructions, perform the further operations comprising inputting data into a non-transitory memory of the computer that is representative of the individual's eating behaviors.

According to an embodiment, the one or more processors are programmed, structured and/or configured to, when executing the instructions, perform the further operations comprising inputting data into a non-transitory memory of the computer that is representative of the individual's nutrition.

According to an embodiment, the one or more processors are programmed, structured and/or configured to, when executing the instructions, perform the further operations comprising inputting data into a non-transitory memory of the computer that is representative of the individual's medications.

According to an embodiment, the one or more processors are programmed, structured and/or configured to, when executing the instructions, perform the further operations comprising inputting data into a non-transitory memory of the computer that is representative of the individual's sleep patterns.

According to an embodiment, the one or more processors are programmed, structured and/or configured to, when executing the instructions, perform the further operations comprising inputting data into a non-transitory memory of the computer that is representative of the individual's physical activity level.

According to an aspect is a computer program product comprising a non-transitory computer readable storage medium containing computer program code, the computer program code when executed by one or more processors causes the one or more processors to perform operations, the computer program code comprising instructions to receive data into the non-transitory computer readable medium of the computer that is representative of the individual's demographics, including weight; periodically receive data into the non-transitory computer readable medium that is representative of the individual's weight loss in units over a first predetermined time interval; periodically receive data into the non-transitory computer readable medium that is representative of the individual's subjective motivation level on a scale graded from lowest motivation to highest motivation level over the first predetermined time interval; calculate data representative of a weight loss slope over a second predetermined interval which is greater than the first time interval; generate a score representing the individual's level of engagement in the weight loss program by summing the data representative of the individual's weight loss in units over a first predetermined time interval, the data representative of the individual's subjective motivation level on a scale graded from lowest motivation to highest motivation level over the first predetermined time interval, and the data representative of a weight loss slope over a second predetermined interval; and generate in the computer and communicating messages to the patient from the computer on a third interval, wherein the messages are customized to the patient based upon the score representing the individual's level of engagement.

According to an embodiment, the first-time interval is one week, the second time interval is four weeks, and the third time interval is daily.

According to an embodiment, the scale representative of the individual's subjective motivation level ranges from 1 to 10 wherein 1 represents the lowest possible motivation and 10 represents the highest possible motivation.

According to an embodiment, the computer program code comprising instructions to perform the further operations comprising the step of transmitting the score representing the individual's level of engagement into a medical record of the individual.

According to an embodiment, the computer program code comprising instructions to perform the further operations comprising inputting data into a non-transitory memory of the computer that is representative of the individual's eating behaviors.

According to an embodiment, the computer program code comprising instructions to perform the further operations comprising inputting data into a non-transitory memory of the computer that is representative of the individual's nutrition.

According to an embodiment, the computer program code comprising instructions to perform the further operations comprising inputting data into a non-transitory memory of the computer that is representative of the individual's medications.

According to an embodiment, the computer program code comprising instructions to perform the further operations comprising inputting data into a non-transitory memory of the computer that is representative of the individual's sleep patterns.

According to an embodiment, the computer program code comprising instructions to perform the further operations comprising inputting data into a non-transitory memory of the computer that is representative of the individual's physical activity level.

These and other aspects of the invention will be apparent from the embodiments described below.

### BRIEF DESCRIPTION OF THE DRAWINGS

The present invention will be more fully understood and appreciated by reading the following Detailed Description in conjunction with the accompanying drawings, in which:

FIG. 1 is a high-level schematic of a system for a system and method for maintaining engagement in a weight loss program, in accordance with an embodiment.

FIG. 2 is a high-level chart of components to a system and method for maintaining engagement in a weight loss program, in accordance with an embodiment.

FIG. 3 is a schematic of a system and method for maintaining engagement in a weight loss program, in accordance with an embodiment.

FIG. 4 is a high-level flow diagram of a system and method for maintaining engagement in a weight loss program, in accordance with an embodiment.

FIG. 5A is a high-level flow chart of a system and method for maintaining engagement in a weight loss program, in accordance with an embodiment.

FIG. 5B is a list of variables used in a system and method for maintaining engagement in a weight loss program, in accordance with an embodiment.

FIGS. 6A and 6B are screen displays on a user interface illustrating the patient intake parameters for a system and method for maintaining engagement in a weight loss program, in accordance with an embodiment.

FIGS. 7A and 7B are screen displays of demographic data input on a user interface, in accordance with an embodiment.

FIGS. 8A, 8B and 8C are screen displays of eating behavior data input on a user interface, in accordance with an embodiment.

FIGS. 9A, 9B and 9C are screen displays of nutrition data input on a user interface, in accordance with an embodiment.

FIGS. 10A and 10B are screen displays of motivational data input on a user interface, in accordance with an embodiment.

FIG. 11 is screen displays of medication data input on a user interface, in accordance with an embodiment.

FIGS. 12A and 12B are screen displays of sleep data input on a user interface, in accordance with an embodiment.

FIGS. 13A, 13B and 13C are screen displays of activity data input on a user interface, in accordance with an embodiment.

FIG. 14 is a user interface screen display for a system and method for maintaining engagement in a weight loss program, in accordance with an embodiment.

FIG. 15 is a screen display of a custom mantra, in accordance with an embodiment.

FIG. 16 is a screen display of a custom daily tip, in accordance with an embodiment.

FIGS. 17A and 17B are screen displays of weight loss tracking data on a user interface, in accordance with an embodiment.

FIG. 18 is a screen displays of weight loss dashboard on a user interface, in accordance with an embodiment.

FIG. 19 is a screen displays of medication data on a user interface, in accordance with an embodiment.

FIG. 20 is a screen displays of daily weight loss data on a user interface, in accordance with an embodiment.

### DETAILED DESCRIPTION OF EMBODIMENTS

The present disclosure describes a system and method for maintaining engagement in a weight loss program.

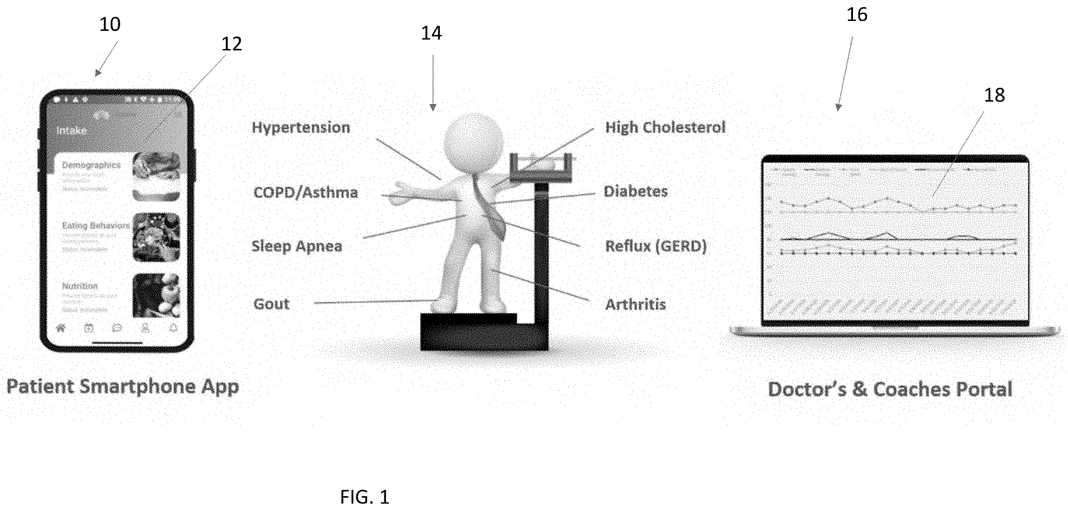

Referring to FIG. 1, in one embodiment, is a high level schematic diagram of the basic components comprising the system and method for maintaining engagement in a weight loss program, namely a patient computing device, such as a smartphone, 10 that includes a user interface 12 (such as a display screen), a plethora of patient data 14, and a medical provider portal 16 that a medical provider can access over a computing device 18, such as a laptop computer. The patient data 14 is stored in the non-transitory memory of a computing device with which the patient computing device 10 and medical provider computing device 18 can access and communicate.

Referring to FIG. 2, the high level construct of the system and method for maintaining engagement in a weight loss program comprises patient intake data 20, a customized patient weight loss plan 22 generated by a computing engine which will be described hereinafter, an evaluation of the patient's medical condition 24, an adjusted customized weight loss plan 26 based on real time data processing of patient data, and an active monitoring of patient progress 28 based on the real time data processing of patient data. All of the data storage and data processing is done on a computing device 30 to which the patient computing device 10 and medical provider computing device 18 can communicate. While it is most typical that the computing device 30 would be a computing system stored in the cloud with communications access achieved via wireless connectivity via a standard communications protocol, other forms of connectivity are possible, including hard-wired connections.

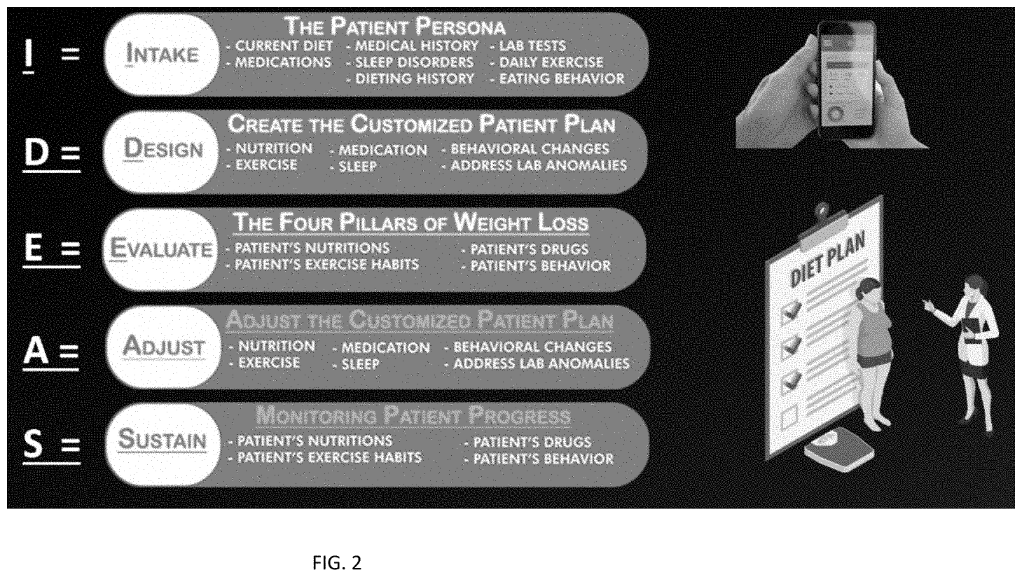

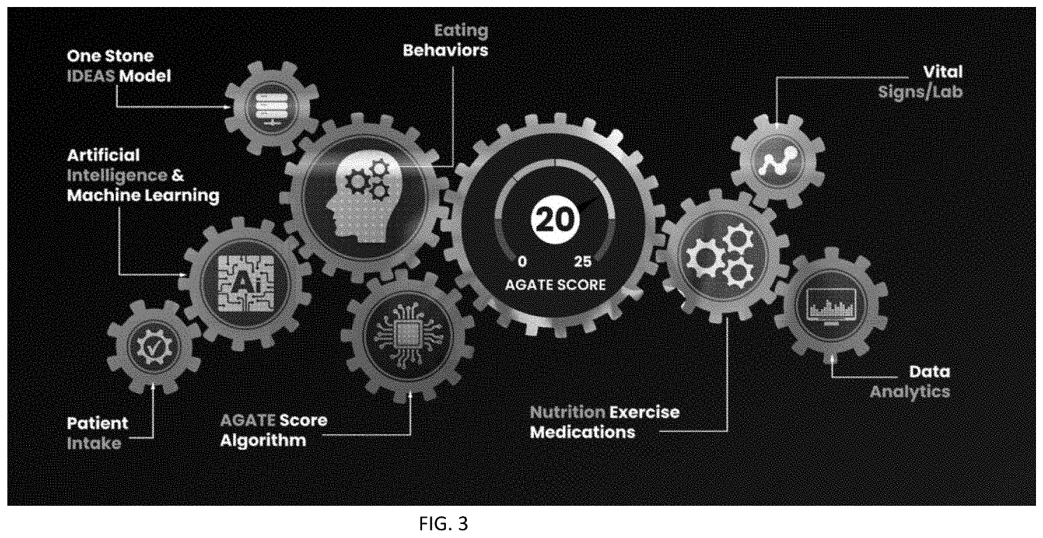

Referring to FIG. 3, a schematic of a system and method for maintaining engagement in a weight loss program is shown symbolically using a series of meshed gears that receive and process weight loss relevant data for purposes of creating a customized weight loss plan and maintaining engagement in the weight loss plan.

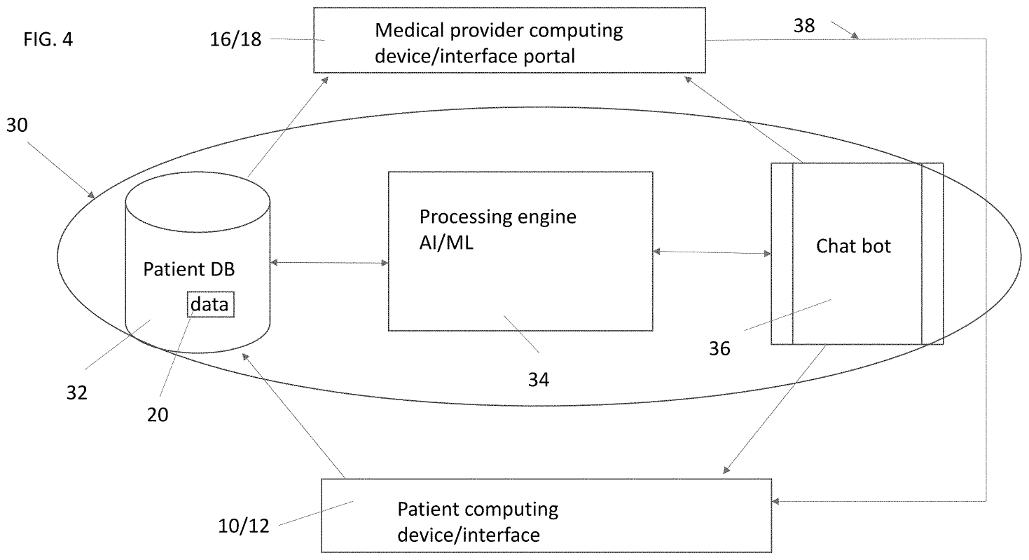

Referring to FIG. 4, the components and flow for the system and method for maintaining engagement in a weight loss program are shown. A computing device 30, preferably stored in a cloud-based architecture, comprises a database of patient data 32, a processing engine 34 that is configured, structured and/or programmed to receive and process that patient data 32 to create a customized weight loss program/engagement mechanism, and a chat bot 36 tool that is programmed, structured and/or configured to interface with the patient computing device 10 and provide customized messaging based upon the data processing done by the processing engine 34. Alternatively, instead of a chat bot providing customized messaging, the medical provider can communicate messaging directly to the patient as shown by communications link 38.

Referring to FIGS. 5A and 5B, the patient first enters data 20 into database 32 in step S1. A listing of variables and data fields considered by the system are provided in FIG. 5B. The data 20 is processed is step S2 using processing engine 34 which is programmed, configured and/or structured to perform machine learning and artificial intelligence processing of the data, as well as calculate a weight loss program score using the formula: Score=WLS+WW+MN, where WLS is the weight loss slope over a predetermined interval (e.g., four weeks) with a quantity of +10 if the slope is negative (e.g., the patient is losing weight), a score of 0 is the slope is flat (e.g., the patient's weight has not changed over the interval), and a score of −10 if the slope is positive (e.g., the patient has gained weight over the interval); WW is the weight loss over a shorter duration (e.g., one week), where a score of +5 points is given if the weight loss is greater than or equal to 0.5 pounds, a score of +3 if the weight loss is between −0.5 and 0.5 pounds over the duration, and a score of 1 if the patient gained a half pound or more over the duration; and MN is the subjective motivation of the patient where the patient enters his/her motivation level on a scale of 1-10 with 1 being the lowest motivation and 10 being the highest motivation relative to maintaining the weight loss plan. Computing device 30/engine 34 interprets the score to reflect the vulnerability of the patient falling from the weight loss program with messaging being generated in accordance with the score. A score of 0-5 indicates the patient is extremely vulnerable to falling off the plan; a score of 6-10 indicates the patient is highly vulnerable to falling of the plan, a score of 11-15 indicates the patient is minimally engaged and somewhat vulnerable to falling off the plan, a score of 16-20 indicates the patient is moderately engaged and motivated to maintain the plan, and a score of 21-25 indicates the patient is highly engaged and motivated to maintain the plan. Based on this score, a chat bot 36 is programmed, configured and/or structured to transmit daily messages to the patient in step S3, and the medical provider is updated as to the patient's status in step S4. Alternatively, to the chat bot providing messaging (or in addition thereto), the medical provider can also provide medical guidance to patient in step S5.

The purpose of the score is to scale the number of coaches/providers needed to keep the patient motivated. The technology permits scaling back on human intervention when they are doing well, and involving more human support when the patient shows signs of struggle and falling off the program. Thus, cost is optimized by minimizing the cost when it is unnecessary, while increasing costs when needed. The technology permits optimization of the cost. Further, when the patient is losing weight, they reverse diseases and can get off or decrease certain weight loss medications. The diseases included in the database when coupled to data associated with the patient's condition permit mediction removal in a more optimized manner.

Finally, the technology keeps the patient engaged with the weight loss program through the optimal assignment of human support or the technological support, and this leads to long-term maintenance of weight loss (as compared to the state of the art wherein the majority of patients regain the weight lost during the program).

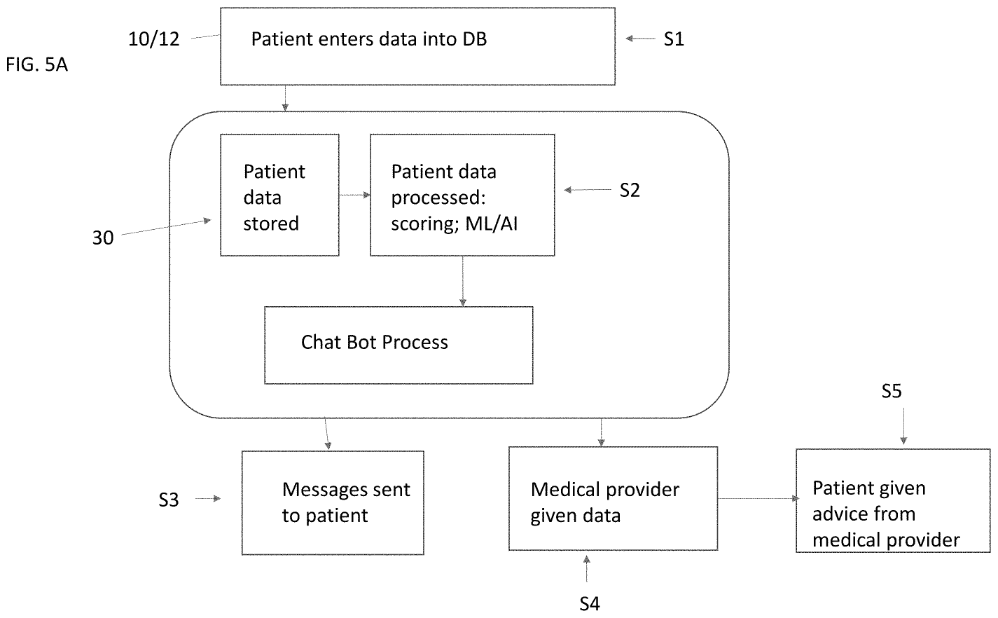

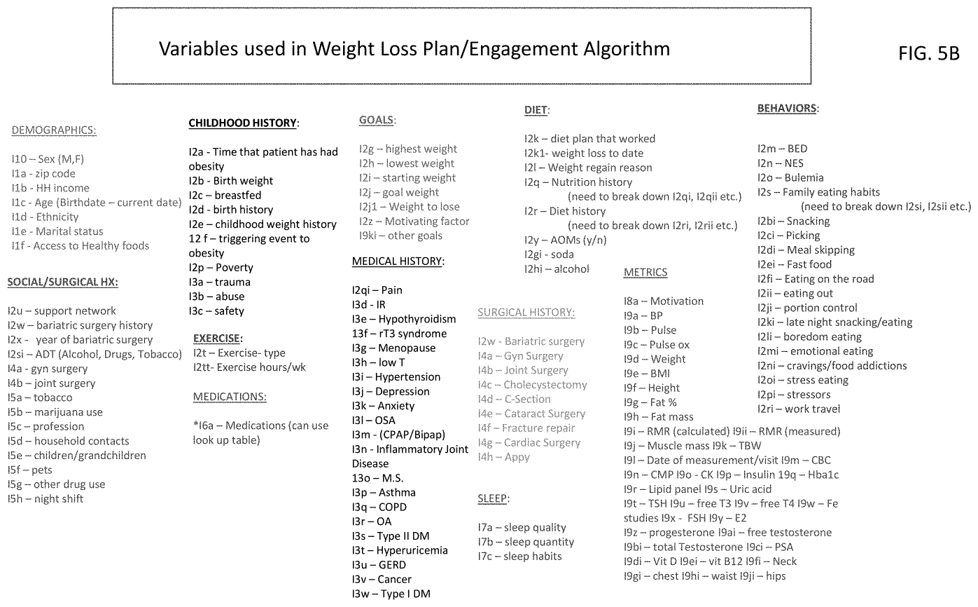

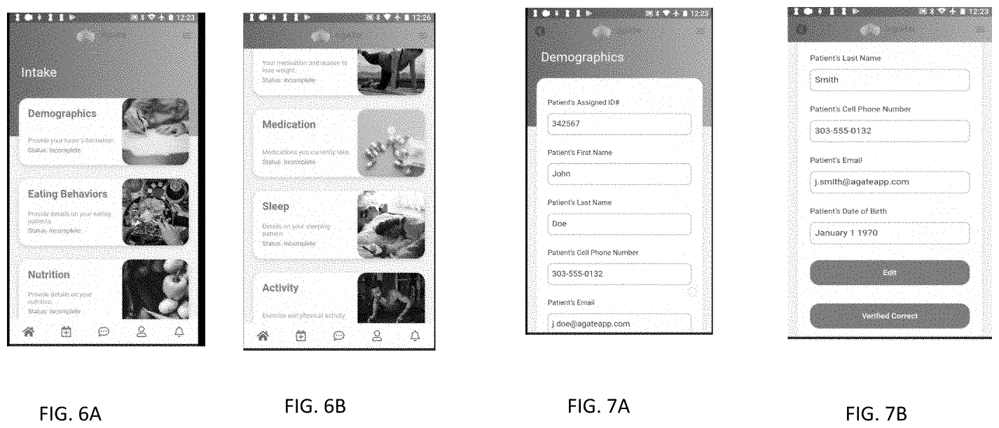

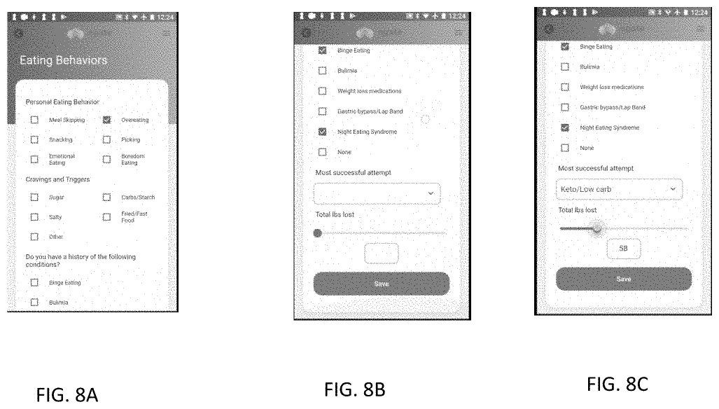

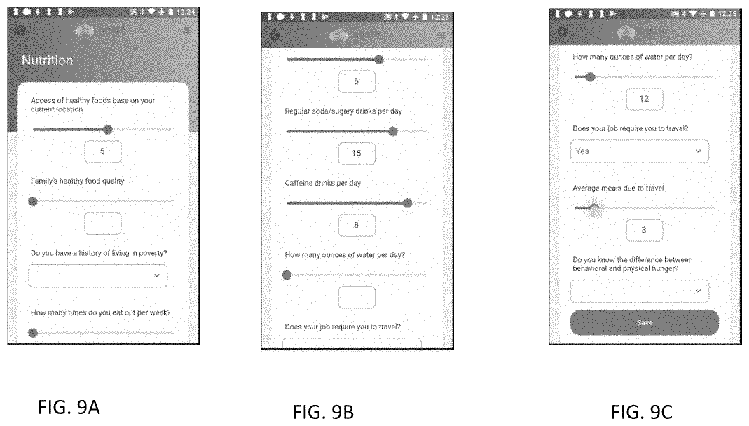

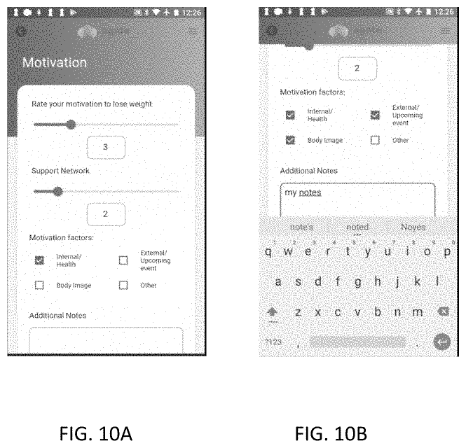

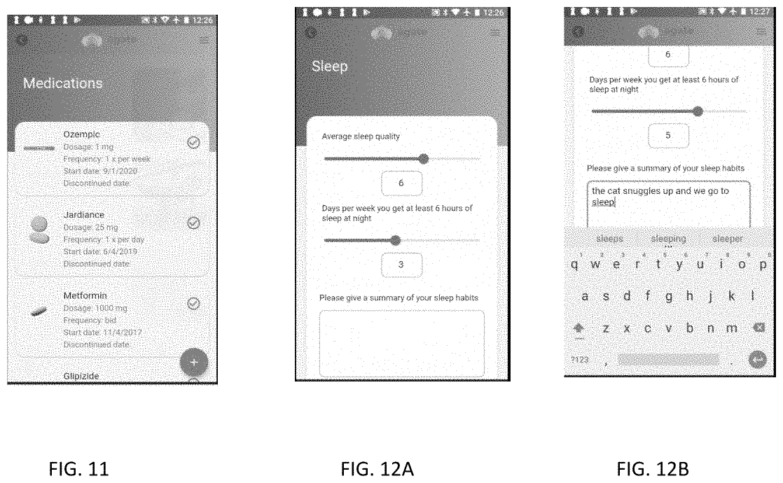

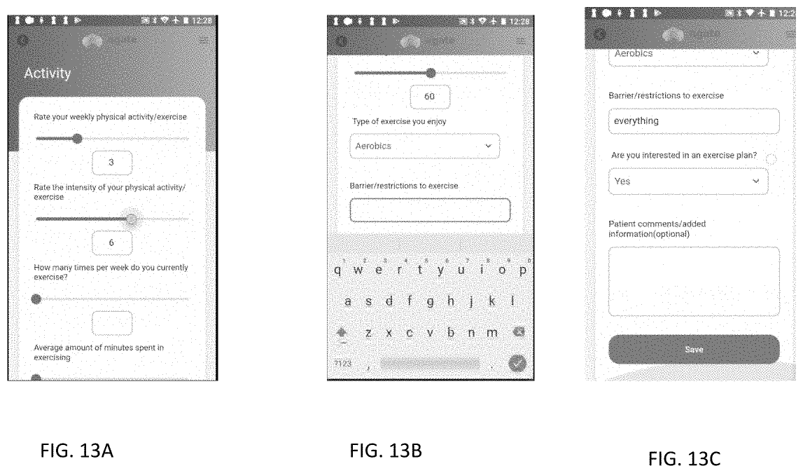

As the system and method for maintaining engagement in a weight loss program is embodied preferably in a smart phone application, with reference to FIGS. 6A and 6B, the initial user interface 12 is shown. A series of data entry points, including demographics, eating behaviors, nutrition, medication, sleep and activity are provided (as is also shown in FIG. 5B). The patient would simply click on the data entry box which will bring up a corresponding user interface, FIGS. 7A and 7B provide the demographic data entry interface, for example, and enter the data, and once all entered and reviewed, verify the data which will save it to database 20. FIGS. 8A-8C show the data entry field for the eating behaviors; FIGS. 9A-9C show the data entry fields for the nutrition data; FIGS. 10A and 10B show the data entry fields for the motivational data; FIG. 11 shows the date entry fields for the medications data (this data entry interface can be populated by pulling medication data from a third party database); FIGS. 122A and 12B show the data entry fields for the sleep data, including a custom text field; and FIGS. 13A-13C show the data entry fields for the activity data. Each data section provides for the patient to save the data to database 20.

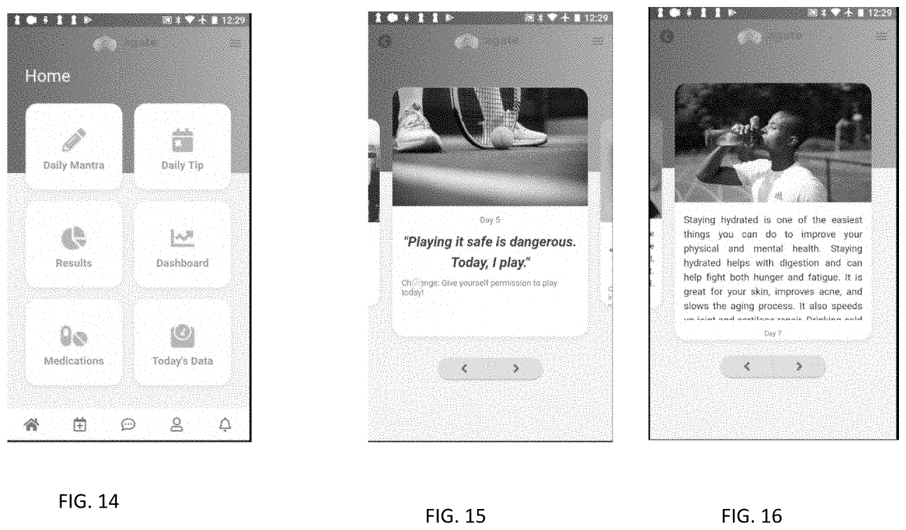

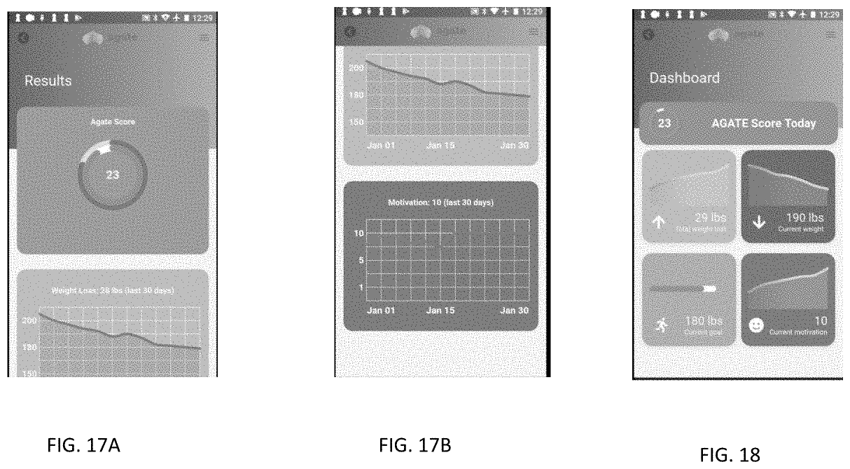

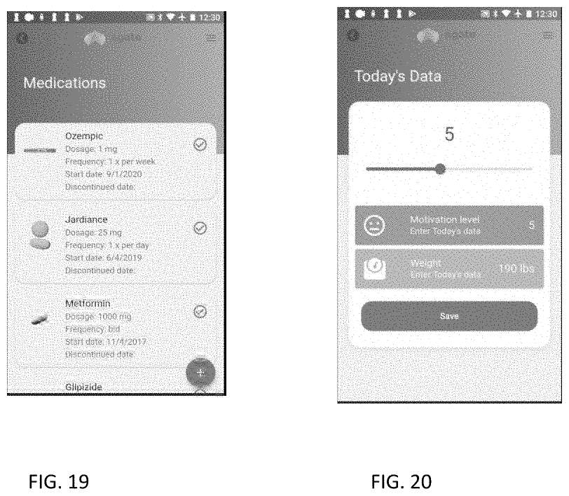

Once the baseline data is entered and the patient begins a weight loss plan, the software application will provide a user interface 12 shown in FIG. 14. A daily mantra will be generated by chatbot 36 and will be customized for patient based upon the data entered, and then transmitted to user device 10 and displayed on user interface 12 as shown in FIG. 15. Also based upon the data entered and processed, the chat bot 36 will generate a custom daily tip for patient intended to help the patient maintain engagement in the weight loss plan (e.g., if the data suggests the patient is not hydrating properly, the daily message can relate to the importance of hydrating), as shown in FIG. 16. Results are also shown to the patient on the interface 12 including the weight loss score, the graph of weight loss over an interval of time, and a graph of the motivation the patient has had over the interval of time, as shown in FIGS. 17A and 17B. Also, a dashboard that provides pointed data points such as the weight loss score as of that day, total weight loss, current weight, current goal, and current motivation level is also displayed on interface 12. As shown in FIG. 18. Finally, a medications data entry tab is provided where the patient can indicate any changes to his/her medications, as shown in FIG. 19, and the data entry point for daily data entry of motivation level and weight is provided as shown in FIG. 20.

While various embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings is/are used. Those skilled in the art will recognize or be able to ascertain using no more than routine experimentation, many equivalents to the specific embodiments described herein. It is, therefore, to be understood that the foregoing embodiments are presented by way of example only and that, within the scope of the appended claims and equivalents thereto, embodiments may be practiced otherwise than as specifically described and claimed. Embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

The above-described embodiments of the described subject matter can be implemented in any of numerous ways. For example, some embodiments may be implemented using hardware, software or a combination thereof. When any aspect of an embodiment is implemented at least in part in software, the software code can be executed on any suitable processor or collection of processors, whether provided in a single device or computer or distributed among multiple devices/computers.

What is claimed is:

1. A computer implemented method of maintaining engagement of an individual in a weight loss program, comprising the steps of:
a. inputting data into a non-transitory memory of the computer that is representative of the individual's demographics, including weight;
b. periodically inputting data into the non-transitory memory of the computer that is representative of the individual's weight loss in units over a first predetermined time interval;
c. periodically inputting data into the non-transitory memory of the computer that is representative of the individual's subjective motivation level on a scale graded from lowest motivation to highest motivation level over the first predetermined time interval;
d. calculating data representative of a weight loss slope over a second predetermined interval which is greater than the first-time interval;
e. generating a score representing the individual's level of engagement in the weight loss program by summing the data representative of the individual's weight loss in units over a first predetermined time interval, the data representative of the individual's subjective motivation level on a scale graded from lowest motivation to highest motivation level over the first predetermined time interval, and the data representative of a weight loss slope over a second predetermined interval; and
f. generating in the computer and communicating messages to the patient from the computer on a third interval, wherein the messages are customized to the patient based upon the score representing the individual's level of engagement.

2. The method of claim 1, wherein the first-time interval is one week, the second time interval is four weeks, and the third interval is daily.

3. The method of claim 1, where the scale representative of the individual's subjective motivation level ranges from 1 to 10 wherein 1 represents the lowest possible motivation and 10 represents the highest possible motivation.

4. The method of claim 1, further comprising the step of transmitting the score representing the individual's level of engagement into a medical record of the individual.

5. The method of claim 1, further comprising the step of inputting data into a non-transitory memory of the computer that is representative of the individual's eating behaviors.

6. The method of claim 1, further comprising the step of inputting data into a non-transitory memory of the computer that is representative of the individual's nutrition.

7. The method of claim 1, further comprising the step of inputting data into a non-transitory memory of the computer that is representative of the individual's medications.

8. The method of claim 1, further comprising the step of inputting data into a non-transitory memory of the computer that is representative of the individual's sleep patterns.

9. The method of claim 1, further comprising the step of inputting data into a non-transitory memory of the computer that is representative of the individual's physical activity level.

10. A system for maintaining engagement of an individual in a weight loss program, comprising:
a. a non-transitory computer readable medium with instruction encoded thereon; and
b. one or more processors programmed, structured and/or configured to, when executing the instructions, perform the operations of:
i. receiving data into the non-transitory computer readable medium of the computer that is representative of the individual's demographics, including weight;
ii. periodically receiving data into the non-transitory computer readable medium that is representative of the individual's weight loss in units over a first predetermined time interval;
iii. periodically receiving data into the non-transitory computer readable medium that is representative of the individual's subjective motivation level on a scale graded from lowest motivation to highest motivation level over the first predetermined time interval;
iv. calculating data representative of a weight loss slope over a second predetermined interval which is greater than the first-time interval;
v. generating a score representing the individual's level of engagement in the weight loss program by summing the data representative of the individual's weight loss in units over a first predetermined time interval, the data representative of the individual's subjective motivation level on a scale graded from lowest motivation to highest motivation level over the first predetermined time interval, and the data representative of a weight loss slope over a second predetermined interval; and
vi. generating in the computer and communicating messages to the patient from the computer on a third interval, wherein the messages are customized to the patient based upon the score representing the individual's level of engagement.

11. The system of claim 10, wherein the first-time interval is one week, the second time interval is four weeks, and the third time interval is daily.

12. The system of claim 10, where the scale representative of the individual's subjective motivation level ranges from 1 to 10 wherein 1 represents the lowest possible motivation and 10 represents the highest possible motivation.

13. The system of claim 10, wherein the one or more processors are programmed, structured and/or configured to, when executing the instructions, perform the further operations comprising the step of transmitting the score representing the individual's level of engagement into a medical record of the individual.

14. The system of claim 10, wherein the one or more processors are programmed, structured and/or configured to, when executing the instructions, perform the further operations comprising inputting data into a non-transitory memory of the computer that is representative of the individual's eating behaviors.

15. The system of claim 10, wherein the one or more processors are programmed, structured and/or configured to, when executing the instructions, perform the further operations comprising inputting data into a non-transitory memory of the computer that is representative of the individual's nutrition.

16. The system of claim 10, wherein the one or more processors are programmed, structured and/or configured to, when executing the instructions, perform the further operations comprising inputting data into a non-transitory memory of the computer that is representative of the individual's medications.

17. The system of claim 10, wherein the one or more processors are programmed, structured and/or configured to, when executing the instructions, perform the further operations comprising inputting data into a non-transitory memory of the computer that is representative of the individual's sleep patterns.

18. The system of claim 10, wherein the one or more processors are programmed, structured and/or configured to, when executing the instructions, perform the further operations comprising inputting data into a non-transitory memory of the computer that is representative of the individual's physical activity level.

19. A computer program product comprising a non-transitory computer readable storage medium containing computer program code, the computer program code when executed by one or more processors causes the one or more processors to perform operations, the computer program code comprising instructions to:
a. receive data into the non-transitory computer readable medium of the computer that is representative of the individual's demographics, including weight;
b. periodically receive data into the non-transitory computer readable medium that is representative of the individual's weight loss in units over a first predetermined time interval;
c. periodically receive data into the non-transitory computer readable medium that is representative of the individual's subjective motivation level on a scale graded from lowest motivation to highest motivation level over the first predetermined time interval;
d. calculate data representative of a weight loss slope over a second predetermined interval which is greater than the first-time interval;
e. generate a score representing the individual's level of engagement in the weight loss program by summing the data representative of the individual's weight loss in units over a first predetermined time interval, the data representative of the individual's subjective motivation level on a scale graded from lowest motivation to highest motivation level over the first predetermined time interval, and the data representative of a weight loss slope over a second predetermined interval; and
f. generate in the computer and communicating messages to the patient from the computer on a third interval, wherein the messages are customized to the patient based upon the score representing the individual's level of engagement.

20. The computer program product of claim 19, wherein the first-time interval is one week, the second time interval is four weeks, and the third time interval is daily.

21. The computer program product of claim 19, where the scale representative of the individual's subjective motivation level ranges from 1 to 10 wherein 1 represents the lowest possible motivation and 10 represents the highest possible motivation.

22. The computer program product of claim 19, wherein the computer program code comprising instructions to perform the further operations comprising the step of transmitting the score representing the individual's level of engagement into a medical record of the individual.

23. The computer program product of claim 19, wherein the computer program code comprising instructions to perform the further operations comprising inputting data into a non-transitory memory of the computer that is representative of the individual's eating behaviors.

24. The computer program product of claim 19, wherein the computer program code comprising instructions to perform the further operations comprising inputting data into a non-transitory memory of the computer that is representative of the individual's nutrition.

25. The computer program product of claim 19, wherein the computer program code comprising instructions to perform the further operations comprising inputting data into a non-transitory memory of the computer that is representative of the individual's medications.

26. The computer program product of claim 19, wherein the computer program code comprising instructions to perform the further operations comprising inputting data into a non-transitory memory of the computer that is representative of the individual's sleep patterns.

27. The computer program product of claim 19, wherein the computer program code comprising instructions to perform the further operations comprising inputting data into a non-transitory memory of the computer that is representative of the individual's physical activity level.
